(19) United States
(12) Patent Application Publication
(10) Pub. No.: US 2026/0224344 A1
(43) Pub. Date: Aug. 6, 2026

(54) PATCH INSERTION TOOL AND RELATED METHODS OF USE

(71) Applicant: Davol Inc., Warwick, RI (US)

(72) Inventor: Ian K. Parker, Bristol, RI (US)

(73) Assignee: Davol Inc., Warwick, RI (US)

(21) Appl. No.: 19/042,166

(22) Filed: Jan. 31, 2025

(51) Int. Cl. A61F 2/00

(52) U.S. Cl. CPC A61F 2/0063; A61F 2002/0072

(57) ABSTRACT

Aspects disclosed herein relate to patch insertion tools for patch bodies used in laparoscopic surgeries. In some embodiments, a patch insertion tool includes a mandrel having a proximal end and a distal end, and a recess in the mandrel. The patch insertion tool includes a proximal lumen extending from the proximal end to the recess. A wire may be configured to extend within the proximal lumen between a retracted position, where the wire is at least partially retracted into the proximal lumen, and an extended position, where the wire may extend at least partially across the recess. A portion of the patch body may be inserted into the recess while the wire is in the retracted position, and the wire may be received into the portion of the patch body when the wire is moved to the extended position to attach the patch body to the mandrel. The patch body may then be furled around the mandrel after attachment.

### FIELD

The field relates to patch insertion tools for delivering patch bodies to surgery sites through trocars, and related methods of use.

### BACKGROUND

Various prosthetic repair patches are employed by surgeons for soft tissue repair and reconstruction, including the repair of anatomical defects such as tissue and muscle hernias. For example, a ventral hernia in the abdominal wall is commonly repaired using a patch formed of a sheet of biocompatible fabric, such as a knitted polypropylene mesh (BARD MESH) or a composite fabric that includes a mesh and an adhesion resistant barrier (COMPOSIX E/X MESH). The patch is typically sutured, stapled or otherwise provisionally anchored in place, over, under or within the defect. Tissue integration with the fabric, such as tissue ingrowth into and/or along the patch, eventually completes the repair. An adhesion resistant barrier, if provided, prevents tissue ingrowth to one or more selected portions of the mesh fabric.

Various surgical techniques may be employed for soft tissue repair, including open, laparoscopic and hybrid (e.g., Kugel procedure). During a laparoscopic procedure, the prosthetic patch may be routed to the surgical site through a slender laparoscopic trocar. The patch is typically collapsed, such as by rolling or folding, into a reduced configuration to facilitate its passage through the narrow trocar. Certain repairs, such as laparoscopic repair of ventral hernias, may require large patches that may be difficult to deliver laparoscopically or through a laparoscopic-like “hybrid” procedure. Devices have been proposed to facilitate delivery of repair fabric into a patient.

### SUMMARY

According to some embodiments, a patch insertion tool is provided. The patch insertion tool comprises a mandrel comprising a proximal end, a distal end, and a length extending between the proximal end and the distal end, a proximal lumen extending through a proximal portion of the length of the mandrel, a recess in the mandrel disposed distal to the proximal lumen, such that a proximal lumen opening is exposed in the recess, and a wire configured to slide within the proximal lumen. The wire is configured to move between a retracted position where the wire is at least partially retracted into the proximal lumen to expose the recess and an extended position where the wire extends across the recess.

According to some embodiments, a method of preparing a patch body for insertion into a trocar is provided. The method comprises inserting a portion of the patch into a recess formed in a mandrel of a patch insertion tool, moving a wire to an extended position to extend the wire across the recess thereby inserting the wire into the portion of the patch body and securing the patch body to the patch insertion tool, and rotating the patch insertion tool about a longitudinal axis of the mandrel to furl the patch body around the mandrel.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows a perspective view of a patch insertion tool according to an embodiment;

FIG. 2 shows a schematic sectional view of a mandrel of a patch insertion tool in a first configuration according to an embodiment;

FIG. 3 shows a schematic sectional view of the mandrel of FIG. 2 in a second configuration according to an embodiment;

FIG. 4 shows a perspective view of a patch insertion tool according to an embodiment;

FIG. 5A shows a top schematic view of a patch insertion tool with a patch body attached in an unfurled configuration according to an embodiment;

FIG. 5B shows a top schematic view of the patch insertion tool and patch body of FIG. 5A with the patch body in a partially furled configuration according to an embodiment;

FIG. 5C shows a top schematic view of the patch insertion tool and patch body of FIG. 5A with the patch body in a fully furled configuration according to an embodiment;

FIG. 6 shows a schematic view of a first step of inserting a patch body into a patient according to an embodiment;

FIG. 7 shows a schematic view of a second step of inserting a patch body into a patient according to an embodiment;

FIG. 8 shows a schematic view of a third step of inserting a patch body into a patient according to an embodiment; and

FIG. 9 shows a schematic view of a fourth step of inserting a patch body into a patient according to an embodiment.

### DETAILED DESCRIPTION

As discussed above, many surgeries, such as hernia repair surgeries, are performed laparoscopically by placing one or more trocars into incisions in a patient, and delivering supplies and/or instruments to the surgery site through the one or more trocars. These hernia repair surgeries often require delivering a patch body to the surgery site through the trocar. It is generally desirable to minimize the diameter of the trocar in order to minimize the size of the incisions made in the patient. However, the patch body needed in order to perform the surgery generally has a relatively large cross-sectional area compared to the inner diameter of the trocar. The patch body is therefore often folded or furled around a patch body insertion tool, inserted into the trocar, and unfurled or unfolded once delivered to the surgery site.

Conventional patch insertion tools generally pinch or clamp the patch body between a pair of tines, and then fold and/or furl the patch body around the tines. However, this approach has several drawbacks. Folding, pinching, and/or clamping the patch body may introduce creases, which may introduce weaknesses into the patch body. Such creases may be particularly undesirable if they are located across the center of the patch body. Additionally, when pinching the patch body between a pair of tines, the patch body is only held in place on the patch insertion tool via friction. It may be difficult to roll the patch body tightly around the patch insertion tool in such a configuration as exerting too much force on the patch body during furling may cause the patch body to slip out of the tines or become misaligned, thus requiring a looser furling resulting in a larger furled diameter of the patch body. Additionally, in such a configuration, because the patch body is only held in place via friction, contact between the patch body and the sides of the trocar during insertion may cause the patch body to become misaligned. As a result of these potential misalignment issues, a larger trocar than desired may generally be used when holding the patch body between a pair of tines. Additionally, such conventional patch insertion tools have no release mechanism to easily release the patch body from the insertion tool, requiring a surgeon to manually release the patch body from the insertion tool at the surgery site using other tools.

The inventor has therefore recognized an advantage to a patch insertion tool configured to selectively attach a portion of the patch insertion tool to a corresponding portion of the patch body. Such a configuration may allow for tight rolling of the patch body around this portion of the patch insertion tool while minimizing the risk of misalignment of the patch body, allowing for the use of a smaller diameter trocar. Such a configuration may also allow for easier release of the patch body from the patch insertion tool after delivering the patch body to the surgery site.

In some embodiments, such a patch insertion tool may comprise a mandrel having a proximal end and a distal end. The mandrel includes a recess and a proximal lumen extending along the length of the mandrel from the proximal end of the mandrel to the recess. The recess includes proximal side closer to the proximal end of the mandrel, and a distal side closer to the distal end of the mandrel. The patch insertion tool also includes a wire configured to slide inside the proximal lumen between a retracted position and an extended position. In the retracted position, the wire is at least partially retracted into the proximal lumen to create a gap between an end of the wire and the distal side of the recess. In the extended position, the wire is slid distally so as toto extend out of the proximal lumen into the recess. It should be understood then when moving the wire to the retracted position, the wire need not be fully retracted into the proximal lumen in some embodiments, such that an end of the wire extends partially into the recess in in the retracted position. Similarly, when moving the wire to the extended position, the wire need not extend fully across the recess to the distal side in some embodiments.

To attach a patch body to the patch insertion tool, the wire is first moved to the retracted position to create the gap between the end of the wire and the distal side of the recess. A portion of the patch body may then be inserted into the gap. The wire is then moved to the extended position to extend across the recess. As the wire is moved to the extended position, the wire is inserted into the portion of the patch body, which is inserted into the gap, securing the patch body to the mandrel. In some embodiments, the wire pierces through the portion of the patch body. In some embodiments, the wire is received into a receiver in the portion of the patch body, such as a sleeve or a loop. In some embodiments, the portion of the patch body is adjacent to an edge of the patch body. In some embodiments, the wire is spaced from a base surface of the recess such that a gap may be present between the wire and the base surface of the recess which may advantageously provide a clearance to allow for easier insertion of the patch body into the gap. In some embodiments, the wire is spaced between 0 mm and 0.75 mm, from the base surface of the recess. In some embodiments, the wire is spaced approximately 0.5 mm from the base surface of the recess. In some embodiments the proximal lumen is offset from a longitudinal axis of the mandrel to allow for maximizing of the clearance between the wire and the base surface while also minimizing a depth of the recess. In some embodiments, the proximal lumen is offset between 0.75 mm and 1.75 mm, from the longitudinal axis of the mandrel.

Once the patch body is attached to the mandrel, the patch insertion tool may be rotated in a furling direction about a longitudinal axis of the mandrel to furl the patch body around the mandrel. Because the patch body is attached to the mandrel, it is possible to furl the patch body more tightly than would be possible with conventional patch insertion tools, resulting in a smaller overall furled diameter. In some embodiments, the patch body may be secured in the furled position (e.g. with one or more elastic bands, an adhesive, or some other fastener) prior to insertion into the trocar. The distal end of the mandrel may then be inserted into the trocar and the furled patch body may be guided to the surgery site. Once the patch body is at the surgery site, the wire may be moved to the retracted position to remove the wire from the patch body and detach the patch body from the patch insertion tool. The patch insertion tool may then be removed from the trocar, leaving the patch body at the surgery site. In some embodiments, the patch insertion tool may be rotated in an unfurling direction once at the surgery site to help facilitate unfurling of the patch body from the mandrel before detaching the patch body from the patch insertion tool. In some embodiments, the patch body may include a self-expanding positioning system attached to the patch body configured to aid in patch deployment, positioning and fixation. Such a self-expanding positioning system may include an elastic frame (e.g. a superelastic nitinol wire) configured to expand when released. Such expansion may allow for easier unfurling of the patch, and may generally hold the patch in the unfurled position during patch attachment.

In some embodiments, the inventor has recognized that it may be desirable for the wire to be supported on both the proximal side and the distal side of the recess when the wire is in the extended position, as such a configuration may limit the likelihood of the wire becoming bent or otherwise misaligned during the furling and insertion processes described above. In some embodiments, the patch insertion tool may include a distal lumen extending along at least a portion of the length of the mandrel from a distal side of the recess towards the distal end of the mandrel. When the wire is moved from the retracted position to the extended position, the wire may slide into the distal lumen. As a result, the wire is supported via contact with the proximal and distal lumens on both the proximal side and distal side of the recess, limiting the likelihood of the distal end of the wire becoming bent or otherwise misaligned during the furling and insertion processes.

The mandrel may be formed using any suitable process. In some embodiments, the mandrel is a unitary body formed from a suitable material such as a metal (e.g. steel (stainless steel, 304 stainless steel, 316 stainless steel, etc.), aluminum, etc.) In such a configuration, the first and/or second lumens may be machined directly into the mandrel. In some embodiments, the mandrel is formed using a core portion and a shell portion configured to be disposed around the core portion. The core portion may include one or more channels which, when the shell portion is disposed around the core portion, form the proximal and/or distal lumens. In some embodiments, the shell portion is formed of a heat-shrinkable material, and is heat shrunk onto the core portion. In some embodiments, the core portion is formed of a polymer (e.g. high density polyethylene, Derlin, etc.), a metal, or any other suitable material. In some embodiments, the shell portion is formed of a metal tube (e.g. a 304 stainless steel tube, a 316 stainless steel tube, etc.), or any other suitable material.

The various components of the patch insertion tool may have any suitable dimensions. In some embodiments, the recess has a length from the proximal side of the recess to the distal side of the recess of between 2 cm and 5 cm In some embodiments, the wire has a diameter between 0.5 mm and 1 mm. In some embodiments, the wire has a diameter of approximately 0.84 mm. In some embodiments, the wire has a diameter of approximately 0.75 mm. In some embodiments, the proximal lumen and/or distal lumen have cross-sectional area(s) of between 0.6 mm² and 1.5mm². In some embodiments, the mandrel has a length sufficient to allow for insertion of a patch body having at least one dimension of 25 cm (e.g. a diameter in the case of a circular or oval patch, or a width or length in the case of a rectangular patch) into a trocar. However, it is contemplated that the length of the mandrel may be selected to allow for any suitably sized or shaped patch body to be inserted into a trocar, as the disclosure is not so limited.

The patch body may be any suitable surgical material suitable for use in various tissue or muscular reinforcement and/or defect correction surgeries such as hernia repair surgeries, anastomosis procedures, an organ wrap procedure, or any other minimally invasive abdominal surgery to an organ which may benefit from a scaffold to promote regenerative soft tissue repair. Examples of materials which may be utilized for the patch body include, but are not limited to, PHASIX Mesh (available from Davol, Inc.), polyglactin (VICRYL—available from Ethicon, Inc.) and polyglycolic acid (DEXON—available from US Surgical, Inc.). Collagen materials such as COOK SURGISIS, available from Cook Biomedical, Inc. may also be used. Non-resorbable materials, including BARD Mesh (available from Davol, Inc.), BARD Soft Mesh (available from Davol, Inc.), SOFT TISSUE PATCH (microporous ePTFE—available from W.L. Gore & Associates, Inc.); SURGIPRO (available from US Surgical, Inc.); TRELEX (available from Meadox Medical); PROLENE and MERSILENE (available from Ethicon, Inc.); and other mesh materials (e.g., available from Atrium Medical Corporation), may be suitable use. It also is contemplated that the patch may be formed from multifilament yarns and that any suitable method, such as knitting, weaving, braiding, molding and the like, may be employed to form the patch body. Thus, a patch body may refer to any suitable mesh, film, or other appropriate structure that may be furled onto a patch insertion tool for insertion and implantation into a subject.

In this application, a number of different material properties/dimensional ranges are provided. However, it should be understood that while those ranges may be beneficial in certain embodiments, other ranges different than those disclosed herein may also be used as the disclosure is not so limited

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

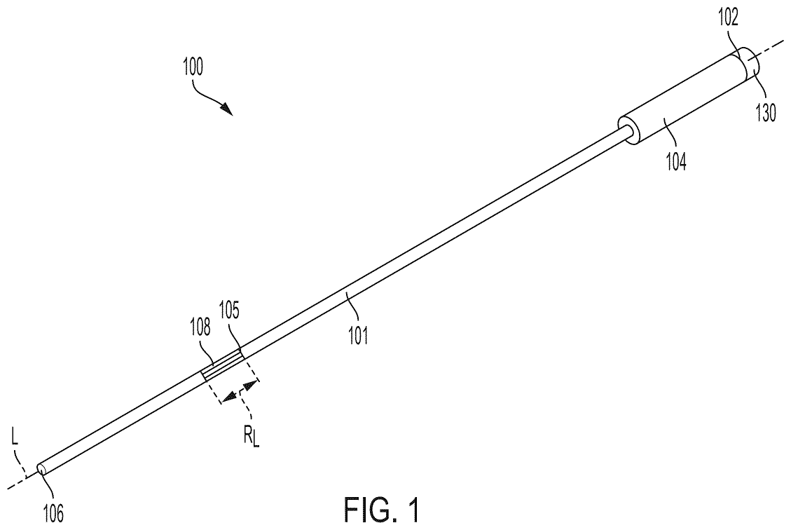

FIG. 1 shows a perspective view of a patch insertion tool according to an embodiment. The patch insertion tool 100 includes a mandrel 101 including a proximal end 102 connected to a handle 104 configured to be grasped by a user's hand when using the patch insertion tool. In some embodiments, the handle 104 may be ergonomically shaped to allow for easier grasping, rather than simply corresponding to the shape of the rest of mandrel 101. The mandrel 101 may extend distally away from the handle 104 to a distal end 106 configured to be inserted into a trocar during use of the patch insertion tool 100. Formed in the mandrel 101 between proximal end 102 and distal end 106 is a recess 108, which is configured to receive a portion of the patch body. The recess 108 extends inwards from a radial edge 105 of the mandrel 101 at least partially through a thickness of the mandrel 101, and extends a length RL along a length of the mandrel 101.

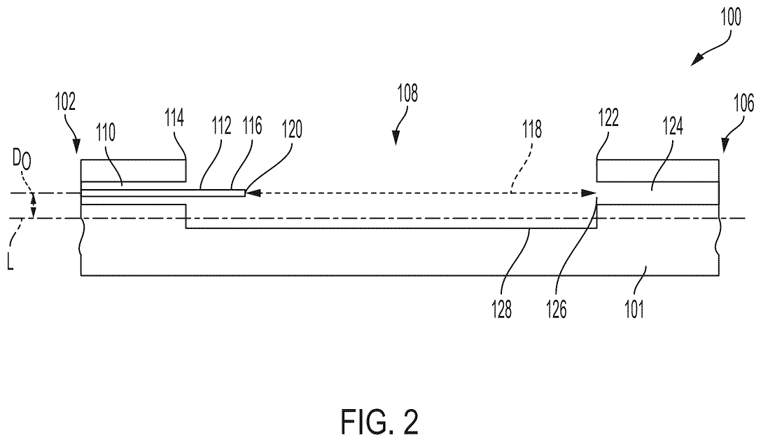

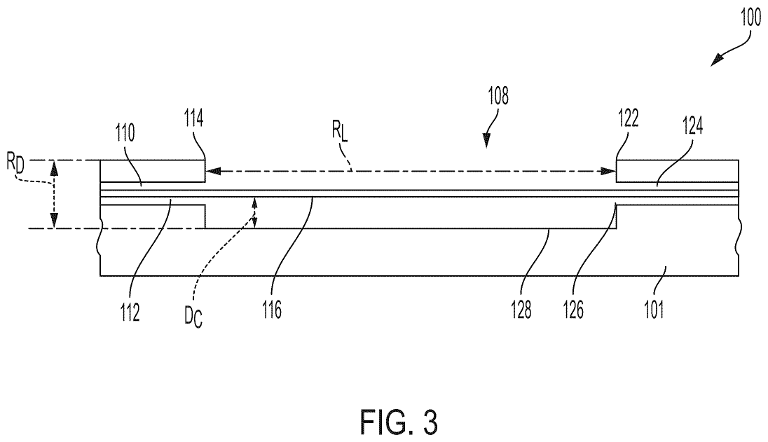

FIG. 2 shows a sectional view of a recess of the patch insertion tool in a first position according to an embodiment. FIG. 3 shows a sectional view of the recess of the patch insertion tool of FIG. 2 in a second position according to an embodiment. The recess 108 may include a proximal side 114, a distal side 122, and a base surface 128. The patch insertion tool 100 may include a proximal lumen 110 extending from proximal end 102 of the mandrel to proximal opening 112 in the proximal side 114 of recess 108. Wire 116 may be configured to slide within the proximal lumen 110 between the retracted position shown in FIG. 2 and the extended position shown in FIG. 3. When wire 116 is in the retracted position, the wire 116 is at least partially retracted into the proximal lumen 110, creating gap 118 between wire end 120 and distal side 122 of the recess 108. A portion of the patch body to be attached to the patch insertion tool 100 may then be inserted into the gap 118. The wire may then be moved to the extended position where the wire 116 extends across gap 118 towards the distal side 122. As wire 116 moves to the extended position, the wire is inserted into the portion of the patch body, securing the patch body to the mandrel 101. In some embodiments, the wire 116 pierces the patch body to secure the patch body to the mandrel 101. In some embodiments, the wire includes a pointed tip to allow for easier piercing of the patch body. In some embodiments, the wire 116 slides into a receiver (e.g. one or more loops and/or sleeves in the patch body) to secure the patch body to the mandrel 101.

In some embodiments, the inventor has recognized that it may be desirable to secure the wire on both the proximal side 114 and the distal side 122 of the recess 108 in order to limit the likelihood of the wire 116 bending during patch furling and insertion, and to limit the likelihood of the patch body unintentionally sliding off of the wire 116 during insertion. The mandrel may therefore include a distal lumen 124 extending from distal opening 126 in the distal side 122 of the recess 108 towards the distal end 106 of the mandrel 101. In some embodiments, the distal lumen extends through the distal end 106, though embodiments in which the distal lumen 124 only extends partially through the distal end portion of the mandrel 101 are also contemplated. When the wire 116 is moved to the extended position, the wire extends into the distal lumen 124. Such a configuration may help support the wire 116 on both the proximal side 114 and the distal side 122 of the recess 108, limiting the likelihood of the wire 116 becoming bent, deflecting under an applied load, and/or the patch body unintentionally sliding off of the mandrel 101 during use of the patch insertion tool 100.

In some embodiments, as best seen in FIG. 3, it may be desirable for the wire 116 to be spaced a clearance distance De from a base surface 128 of the recess 108, in order to allow sufficient clearance for the patch body to be inserted into the recess 108. In some embodiments, the clearance distance De is between 0 mm and 0.75 mm. In some embodiments, the clearance distance De is approximately 0.5 mm. In some embodiments, it may be desirable to minimize a depth RD of recess 108 into the mandrel 101, in order to ensure the mandrel 101 remains sufficiently stiff during use and to avoid buckling of the mandrel 101 during use. In some embodiments, the depth Rp of the recess 108 is between 1 mm and 2 mm. In some embodiments, the depth Rp of the recess 108 is approximately 1.5 mm.

In some embodiments, as best seen in FIG. 2 the proximal lumen 110 and/or the distal lumen 124 may be offset a distance Do from longitudinal axis L of the mandrel 101 to allow for sufficient clearance between the wire 116 and the base surface 128 while still minimizing the depth of recess 108. In some embodiments, offset distance Do may be between 0.75 mm and 1.75 mm. In some embodiments, the recess 108 has a length RL between 2 cm and 5 cm. In some embodiments, the proximal lumen 110 and/or distal lumen 124 may extend approximately parallel to the longitudinal axis L. In some embodiments, the longitudinal axis L passes through a centroid of the mandrel 101.

While the base surface 128 is shown as a planar surface extending approximately parallel to the longitudinal axis L, it should be understood that the base surface may be unaligned with the longitudinal axis L, or may be a non-planar surface, as the disclosure is not so limited.

Additionally, as best seen in FIG. 2, while the depth of the recess is shown as extending through the longitudinal axis L, such that base surface 128 is “below” the longitudinal axis L, it should be understood that in some embodiments, the depth of the recess does not extend through the longitudinal axis, such that the base surface is “above” the longitudinal axis L, as the disclosure is not so limited.

The mandrel 101 may have any suitable size to enable use of the patch insertion tool 100 in a laparoscopic surgery. In some embodiments, the length of the mandrel 101 (excluding the handle) may be between 30 cm and 40 cm. In some embodiments, the diameter of the mandrel may be between 3 mm and 5 mm. In some embodiments, the mandrel 101 has a substantially constant diameter from the handle 104 to the distal end 106, such that the mandrel 101 comprises a substantially cylindrical shape. In some embodiments, the mandrel 101 is tapered from the proximal end 102 to the distal end 106. The wire 116 may be made out of any suitable material for use in a laparoscopic surgery, such as nitinol, stainless steel, or any other suitable material. In some embodiments, the wire 116 has a diameter between 1 mm and 5 mm. In some embodiments, the wire 116 has a length such that the wire does not extend out of distal lumen 124 when the wire is in the fully extended position in embodiments where the distal lumen extends through distal end 106.

In some embodiments, it may be desirable to allow for easier manipulation of the wire 116 between the extended and retracted positions. Therefore, as seen in FIG. 1, the patch insertion tool 100 may include a wire handle 130 connected to the wire 116 at a proximal end portion 102 of the mandrel 101. The wire handle 130 may be ergonomically shaped to allow for a user to more easily manipulate the wire 116. The wire handle 130 may also serve as a stopper to prevent the wire 116 from sliding fully into the proximal lumen 110 during use. The wire handle 130 may be configured to move proximally along longitudinal axis L to move the wire 116 to the retracted position, and may be configured to move distally relative to longitudinal axis L to move the wire to the extended position. In some embodiments, the wire handle 130 may be configured to lock into the handle 104 when the wire 116 is in the extended position (e.g. via a twist locking mechanism, or any other suitable conventional mechanism), in order to limit the likelihood of the wire 116 inadvertently moving to the retracted position.

In some embodiments, the mandrel 101 is formed of a unitary body, and recess 108, proximal lumen 110, and/or distal lumen 124 are machined from the unitary body. In some embodiments, the mandrel 101 comprises a multilayer construction.

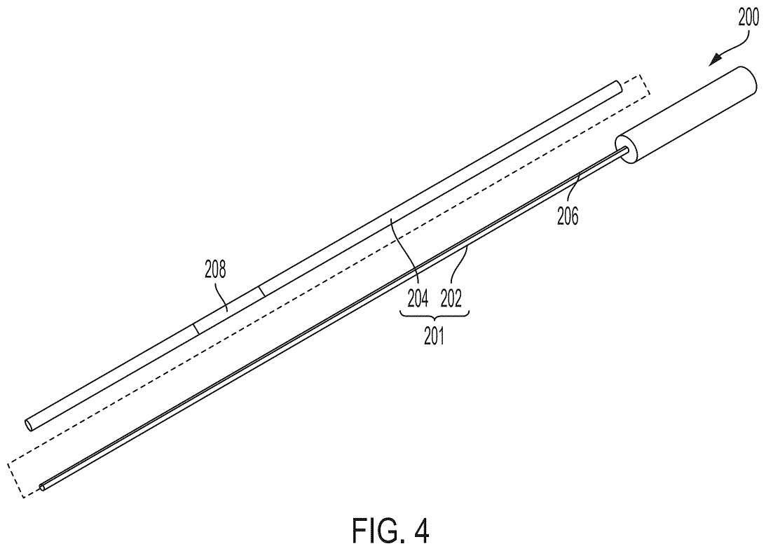

FIG. 4 shows an exploded perspective view of a patch insertion tool 200 with a mandrel 201 having a multilayer construction according to an embodiment. The mandrel 201 comprises a core portion 202 and a shell portion 204. The core portion 202 comprises a groove 206 extending along the length of the mandrel 201. The shell portion 204 includes a cutout 208. When the shell portion 204 is disposed over the core portion 202, the cutout 208 aligns with groove 206 to form a recess. In some embodiments, the core portion 202 already includes a recess, and the cutout 208 aligns with this recess to allow for insertion of the patch body. The shell portion 202 also covers the exposed portion of groove 206 to form proximal and distal lumens on either side of cutout 208, which may receive a wire similar to any embodiment discussed above. In some embodiments, the shell portion 202 is formed of a heat shrinkable material in order to allow for easer insertion of the core portion 202 into the shell portion 204. In some embodiments, the core portion is formed of a metal material, a polymer etc. In some embodiments, the shell portion is formed of a metal tube.

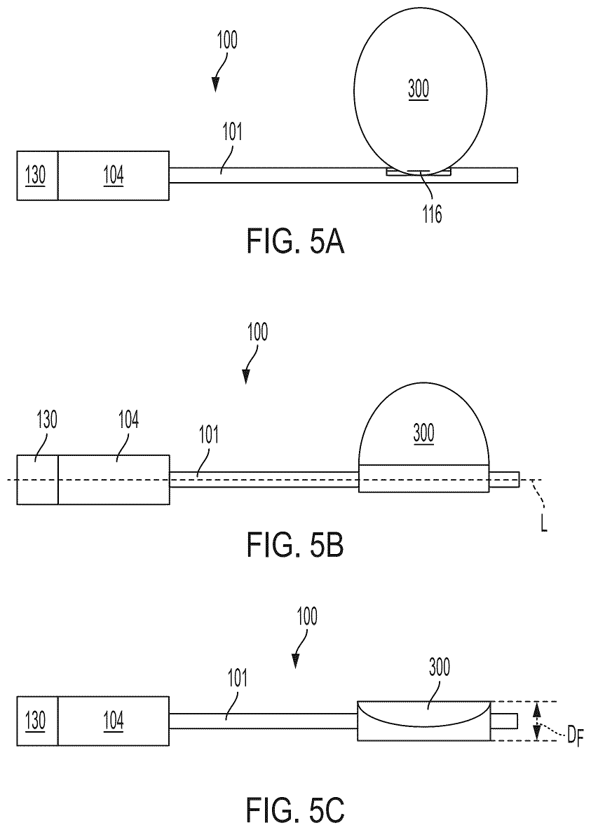

FIGS. 5A-5C show top schematic views of patch insertion tool 100 in various states of furling according to an embodiment. In use, a patch body may be secured to the patch insertion tool 100 by moving the wire 116 to the retracted position to expose the gap 118, inserting the portion of the patch body 300 into the gap 118, and moving the wire to the extended position to insert wire 116 into a portion of the patch body 300 (e.g. by piercing the wire through an outer edge of the patch body, by inserting the wire into a receiver in the patch body, etc.). The patch body attached to the patch insertion tool using the above described method may be seen best in FIG. 5A. Once the patch body 300 is secured to the patch insertion tool 100, the mandrel 101 may be rotated about longitudinal axis L in order to furl the patch body 300 around the mandrel 101, as best seen in FIG. 5B. Because the patch body 300 is secured to the mandrel 101, it is possible to furl the patch body 300 more tightly than would be possible with other conventional patch insertion tools. Once, the patch body 300 is fully furled about the mandrel 101, as seen in FIG. 5C, the furled patch body diameter DF may be small enough to allow for insertion into a trocar. In some embodiments, the furled patch body diameter DF is between 4.5 cm and 5 cm. In some embodiments, the patch body 300 is secured in the furled configuration (e.g. via an elastic band, adhesive, clasp, or other suitable fastener) to limit the likelihood of the patch body 300 from unfurling during insertion.

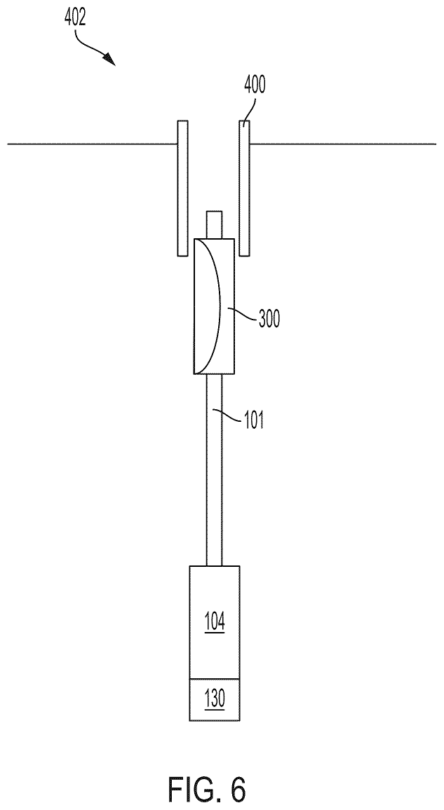

As elaborated on below, FIGS. 6-9 show schematic representations of a process of inserting a patch body into a patient according to an embodiment.

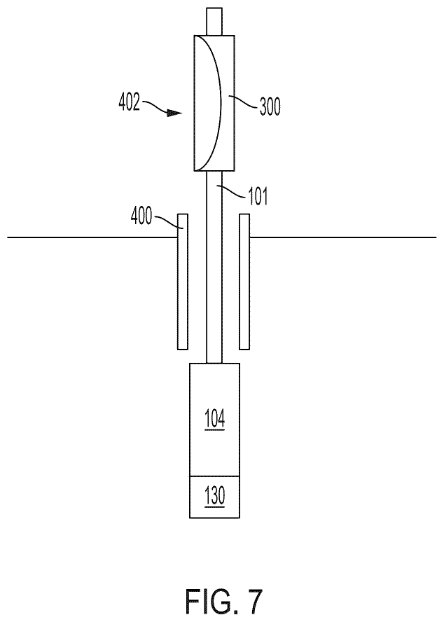

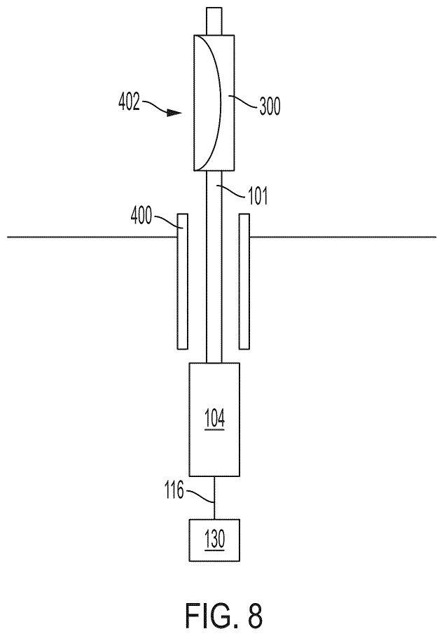

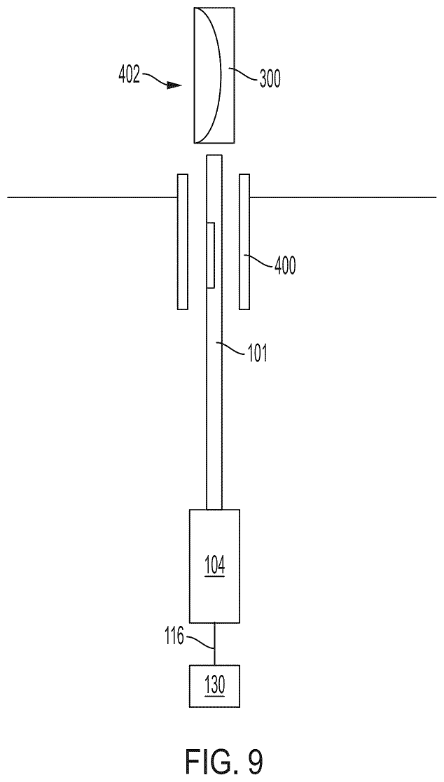

In order to insert the patch body 300 via the patch insertion tool 100. One or more trocars 400 may be placed in a patient's body via any suitable surgical method. The trocars may have any suitable inner diameter, such as between 5 mm and 15 mm. A patch insertion tool 100 with a furled patch body 300 may then be inserted into the patient's body through a trocar 400, as seen in FIG. 6, and guided to the surgery site 402, as seen in FIG. 7. The wire handle 130 may then be actuated by the operator in order to retract the wire and release the patch body 300 from the mandrel 101, as seen in FIG. 8. The patch body 300 may then be slid off of the mandrel 101 as the patch insertion tool 100 is removed from the patient's body via trocar 400, leaving the patch body 300 at the surgery site 402 in a furled configuration, as seen in FIG. 9. In some embodiments, the patch body 300 is unfurled from the mandrel 101 before being detached from the patch insertion tool 100. In some embodiments, the patch body 300 includes a self-expanding positioning system to unfurl the patch body 300 at the surgery site 402.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

Accordingly, the foregoing description and drawings are by way of example only.

1. A patch insertion tool comprising:
a mandrel comprising a proximal end, a distal end, and a length extending between the proximal end and the distal end;
a proximal lumen extending through a proximal portion of the length of the mandrel;
a recess in the mandrel disposed distal to the proximal lumen, such that a proximal lumen opening is exposed in the recess; and
a wire configured to slide within the proximal lumen, the wire configured to move between a retracted position where the wire is at least partially retracted into the proximal lumen to expose the recess and an extended position where the wire extends across the recess.

2. The patch insertion tool of claim 1, wherein the wire is configured to be inserted into a patch body at least partially disposed in the recess to secure the patch body to the patch insertion tool when the wire is moved from the retracted position to the extended position, and wherein the wire is configured to release the patch body from the patch insertion tool when the wire is moved from the extended position to the retracted position.

3. The patch insertion tool of claim 2, wherein the wire comprises a pointed tip configured to pierce into a portion of the patch body.

4. The patch insertion tool of claim 2, further comprising the patch body, and wherein the wire is inserted along an outer edge of the patch body.

5. The patch insertion tool of claim 4, wherein the patch body is a patch body for use in a hernia repair surgery.

6. The patch insertion tool of claim 4, wherein the patch body comprises at least one receiver configured to receive the wire to selectively attach the patch body to the patch insertion tool.

7. The patch insertion tool of claim 1, further comprising a distal lumen extending into a distal portion of the length of the mandrel, the distal lumen comprising a distal lumen opening exposed in the recess, wherein the wire is configured to be retracted out of the distal lumen in the retracted position and to extend into the distal lumen in the extended position.

8. The patch insertion tool of claim 7, wherein the distal lumen extends through the distal end of the mandrel, and wherein the wire has a length such that the wire does not extend out of the distal end of the mandrel when the wire is in the extended position.

9. The patch insertion tool of claim 7, wherein the recess comprises a base surface extending along the length of the mandrel between the proximal lumen opening and the distal lumen opening.

10. The patch insertion tool of claim 7, wherein the mandrel comprises a longitudinal axis extending along the length through a centroid of the mandrel, and wherein the proximal lumen and the distal lumen extend approximately parallel to the longitudinal axis.

11. The patch insertion tool of claim 10, wherein the proximal lumen and the distal lumen are offset from the longitudinal axis.

12.-15. (canceled)

16. The patch insertion tool of claim 1, wherein the mandrel is a unitary body.

17. The patch insertion tool of claim 1, wherein the mandrel comprises a core portion formed of a first material and shell portion formed of a second material.

18. The patch insertion tool of claim 17, further comprising a distal lumen extending through a distal portion of the length of the mandrel, the distal lumen comprising a distal lumen opening exposed in the recess, wherein the wire is configured to be retracted out of the distal lumen in the retracted position and to extend into the distal lumen in the extended position, wherein the core portion comprises a proximal groove and a distal groove, and wherein the shell portion is configured to cover the proximal groove to form the proximal lumen and to cover the distal groove to form the distal lumen.

19. The patch insertion tool of claim 17, wherein the shell portion is formed of a heat shrinkable material.

20. (canceled)

21. The patch insertion tool of claim 1, further comprising a handle having a wire handle connected to the wire and disposed at the proximal end of the mandrel.

22. The patch insertion tool of claim 21, wherein the wire handle is configured to selectively lock into the handle when the wire is in the extended position.

23. A method of preparing a patch body for insertion into a trocar, the method comprising:
inserting a portion of the patch into a recess formed in a mandrel of a patch insertion tool;
moving a wire to an extended position to extend the wire across the recess thereby inserting the wire into the portion of the patch body and securing the patch body to the patch insertion tool; and
rotating the patch insertion tool about a longitudinal axis of the mandrel to furl the patch body around the mandrel.

24. The method of claim 23, further comprising moving the wire to a retracted position where the wire is at least partially retracted into a proximal lumen to expose the recess in the retracted position.

25. The method of claim 24, further comprising inserting the furled patch body and mandrel into a patient's body via a trocar.

26. The method of claim 25, further comprising moving the wire to the retracted position to release the patch body from the patch insertion tool.

27. The method of claim 24, wherein the mandrel comprises a distal lumen extending through a distal portion of the mandrel, wherein moving the wire to the retracted position retracts the wire out of the distal lumen, and wherein moving the wire to the extended position extends the wire into the distal lumen.

28. The method of claim 27, wherein the mandrel comprises a longitudinal axis extending along a length of the mandrel through a centroid of the mandrel, and wherein the proximal lumen and the distal lumen extend approximately parallel to the longitudinal axis.

29. The method of claim 28, wherein the proximal lumen and the distal lumen are offset from the longitudinal axis.

30. (canceled)

31. The method of claim 23, wherein inserting the wire into the portion of the patch body comprises piercing a portion of the patch adjacent to an edge of the patch body.

32. The method of claim 23, wherein inserting the wire into the portion of the patch body comprises inserting the wire into a receiver of the patch.

33.-36. (canceled)

37. The method of claim 23, wherein the mandrel comprises a core portion formed of a first material and shell portion formed of a second material.

38. The method of claim 37, wherein the core portion of the mandrel comprises a proximal groove and a distal groove, and wherein the shell portion is configured to cover the proximal groove to form a proximal lumen and to cover the distal groove to form a distal lumen.

39. (canceled)

40. (canceled)

41. The method of claim 23, wherein the patch insertion tool further comprises a handle having a wire handle connected to the wire and disposed at a proximal end of the mandrel.

42. The method of claim 23, wherein the patch body is a patch body for a hernia repair surgery.
